(19) United States
(12) Patent Application Publication
(10) Pub. No.: US 2026/0314965 A1
(43) Pub. Date: Oct. 15, 2026

(54) TISSUE-OXYGENATION MONITORING SYSTEM AND METHOD

(71) Applicants: The Trustees of Dartmouth College, Hanover, NH (US); Wisconsin Alumni Research Foundation, Madison, WI (US)

(72) Inventors: Arthur Felix Petusseau, Hanover, NH (US); Brian Pogue, Madison, WI (US)

(21) Appl. No.: 19/647,844

(22) Filed: Apr. 14, 2026

Related U.S. Application Data
(60) Provisional application No. 63/789,161, filed on Apr. 15, 2025.

(51) Int. Cl. A61B 5/1455; A61B 5/00; A61N 5/10

(52) U.S. Cl. CPC A61B 5/14556; A61B 5/4839; A61N 5/1077

(57) ABSTRACT

A tissue-oxygenation monitoring method includes illuminating tissue that contains protoporphyrin IX (PPIX) with an illumination having spectral components within a fluorescence excitation band of the PPIX, thereby generating a delayed fluorescence (DF) signal propagating from the tissue. The method also includes detecting the DF signal and determining a tissue oxygenation state at least in part from the detected DF signal.

### CROSS-REFERENCE TO RELATED APPLICATION

This application claims the benefit of U.S. Provisional Application No. 63/789,161, filed on 15 Apr. 2025, the disclosure of which is incorporated herein by reference in its entirety.

### STATEMENT REGARDING FEDERALLY SPONSORED RESEARCH OR DEVELOPMENT

This invention was made with government support under U01 CA260446 awarded by the National Institutes of Health. The government has certain rights in the invention.

### BACKGROUND

Current methods for measuring tissue oxygenation during radiotherapy typically rely on indirect techniques, including blood oxygenation measurements or imaging systems that do not provide real-time, continuous feedback. While methods like positron emission tomography (PET) and magnetic resonance imaging (MRI) have been employed to assess tumor oxygenation, these methods are often expensive, have limited spatial resolution, or require significant post-processing time. One of the gold standard methods for measuring oxygen levels is pulse oximetry; however, this technique primarily measures blood oxygen saturation, which may not accurately reflect the actual tissue oxygen concentration. Additionally, pulse oximetry is highly sensitive to tissue optical properties, such as skin pigmentation, making it less reliable for patients with darker skin tones.

### SUMMARY OF THE EMBODIMENTS

The lack of a real-time, non-invasive method to monitor tissue oxygenation during radiotherapy is a primary problem in the field. Embodiments disclosed herein address this problem. Tumor oxygenation is critical for the effectiveness of radiation therapy, as tumors with low oxygen levels (hypoxia) are more resistant to radiation. However, existing techniques for monitoring oxygenation are either invasive, require specialized equipment, or provide indirect measurements that do not allow for immediate clinical decision-making.

Embodiments disclosed herein include a method for assessing tissue oxygenation during radiotherapy by utilizing aminolevulinic acid (ALA) to induce protoporphyrin IX (PpIX) and measuring its delayed fluorescence (DF). The method enables real-time, non-invasive quantification of oxygen levels in tissue, which is particularly relevant for optimizing radiotherapy treatment, including FLASH radiotherapy.

The method involves administering ALA to a patient, leading to the accumulation of PpIX in tissues. Upon excitation, PpIX exhibits delayed fluorescence, which is dependent on tissue oxygenation levels. By capturing and analyzing PpIX DF signals, embodiments provide a direct measurement of tissue oxygenation, enabling improved treatment monitoring and adaptation. Oxygen is a key factor in tissue responsiveness to radiotherapy, making disclosed embodiments highly valuable for optimizing treatment outcomes.

This approach is particularly significant for FLASH radiotherapy, where ultra-high dose rate (UHDR) radiation is believed to have an oxygen-dependent biological effect. By integrating PpIX DF imaging into radiotherapy workflows, clinicians can assess the oxygenation status of tumors and surrounding tissues, optimizing treatment planning and delivery.

Unlike conventional methods, embodiments disclosed herein enable real-time, spatially resolved oxygenation mapping during radiotherapy. ALA-PpIX is an FDA-approved contrast agent that is well-tolerated by the human body. As such, it represents the only method for measuring intracellular tissue oxygen concentration in humans with no associated risks, using a well-established and widely recognized drug. Embodiments herein may be tailored for assessing oxygen levels in FLASH therapy, a rapidly emerging technique in cancer treatment.

The key differentiation of disclosed embodiments is the real-time, non-invasive ability to image tissue oxygenation using ALA-induced PpIX DF, a process that has not been fully explored for this specific application in the context of radiotherapy. Furthermore, the ability to monitor oxygen levels during UHDR RT with high accuracy is a crucial advancement not offered by current imaging technologies.

By using ALA-induced PpIX and its delayed fluorescence, embodiments disclosed herein offer one or more of the following advantages:
Real-time monitoring: The technology allows clinicians to measure tissue oxygen levels in real-time during treatment, providing immediate feedback and the ability to adjust treatment plans if necessary.
Non-invasive approach: Unlike invasive oxygen measurements or biopsy-based approaches, this method is non-invasive and integrates seamlessly with radiotherapy procedures.
No additional risks: As ALA is an FDA-approved contrast agent, it introduces no new risks to patients and utilizes a well-established substance that is already widely recognized for clinical use.
Broad applicability: The technology may be applied across various radiotherapy types, including standard and UHDR radiotherapy, making it versatile and suitable for a wide range of clinical settings.
Enhanced treatment planning: By providing accurate tissue oxygenation data, embodiments can help optimize treatment plans, potentially improving therapeutic outcomes by targeting hypoxic tumor regions that may otherwise be resistant to radiation.
   In summary, embodiments improve upon existing technologies by offering a real-time, non-invasive, and clinically viable method to monitor tissue oxygenation during radiotherapy, providing immediate insights into treatment efficacy and enabling better-informed clinical decisions.

In a first aspect, a tissue-oxygenation monitoring method includes illuminating tissue that contains protoporphyrin IX (PPIX) with an illumination having spectral components within a fluorescence excitation band of the PPIX, thereby generating a delayed fluorescence (DF) signal propagating from the tissue. The method also includes detecting the DF signal and determining a tissue oxygenation state at least in part from the detected DF signal.

In a second aspect, a tissue-oxygenation monitoring system includes a light source, a photodetector, and processing circuitry. The light source is configured to illuminate tissue with illumination having spectral components within a fluorescence excitation band of protoporphyrin IX (PPIX), thereby generating a delayed fluorescence (DF) signal propagating from the tissue. The photodetector is configured to detect the DF signal. The processing circuitry is communicatively coupled to the photodetector and is configured to determine, at least in part from the detected DF signal, an oxygenation state indicative of oxygenation of the tissue.

### BRIEF DESCRIPTION OF THE DRAWINGS

FIG. 1 is a schematic of a tissue-oxygenation monitoring system, in an embodiment.

FIG. 2 is a flowchart illustrating an embodiment of a tissue-oxygenation monitoring method, which may be implemented by the system of FIG. 1.

FIG. 3 is a schematic of a tissue-oxygenation monitoring system, which is an embodiment of the system of FIG. 1.

FIGS. 4A-4D depict the outcomes of UHDR irradiation of mice with various doses.

FIGS. 5A and 5B show that tissue oxygen depletion increases as a function of initial pO2 and eventually saturates at higher initial oxygen levels.

FIGS. 6A-6D show an example dependence of ΔpO2 on the initial pO2 values, as measured using a PpIX ratiometric signal generated by an embodiment of the system of FIG. 1. FIG. 6E shows a distribution optical probes in the murine model and locations of ROIs used for collection of data plotted in FIGS. 6A-6D.

FIGS. 7A and 7B illustrate the relationship between oxygen depletion measured using both PdG4 and PpIX probes.

### DETAILED DESCRIPTION OF THE EMBODIMENTS

### 1. Tissue-Oxygenation Monitoring System

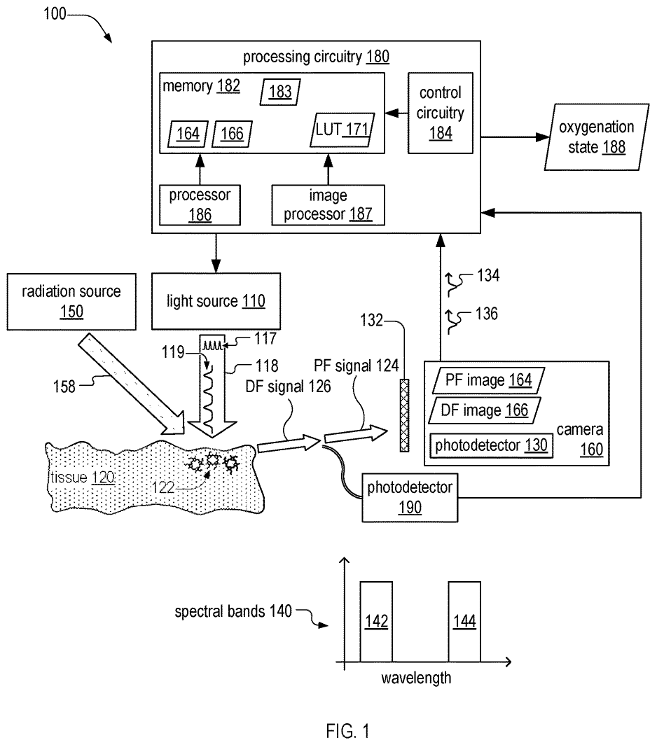

FIG. 1 is a schematic of a tissue-oxygenation monitoring system 100, herein after system 100. System 100 includes a light source 110, a photodetector 130, and a processing circuitry 180. Processing circuitry 180 is communicatively coupled to one or both of light source 110 and photodetector 130, and may include one or both of a memory 182 and a processor 186. Light source 110 may include LED and/or a laser.

Memory 182 may be transitory and/or non-transitory and may include one or both of volatile memory (e.g., SRAM, DRAM, computational RAM, other volatile memory, or any combination thereof) and non-volatile memory (e.g., FLASH, ROM, magnetic media, optical media, other non-volatile memory, or any combination thereof). Part or all of memory 182 may be integrated into processor 186. Memory 182 stores software 183 that includes non-transitory machine-readable instructions. When executed by processor 186, software 183 causes processor 186 to implement one or more functions of system 100 as described herein. Software 183 may be, or include, firmware.

Processor 186 represents any type of circuit or integrated circuit capable of performing logic, control, and input/output operations. For example, processor 186 may include one or more of a microprocessor with one or more central processing unit (CPU) cores, a graphics processing unit (GPU), a digital signal processor (DSP), a field-programmable gate array (FPGA), a system-on-chip (SoC), a microcontroller unit (MCU), and an application-specific integrated circuit (ASIC). Processor 186 may also include a memory controller, bus controller, and other components that manage data flow between processor 186 and memory 182.

Memory 182 stores software 183 that includes non-transitory machine-readable instructions. When executed by processor 186, software 183 causes processor 186 to implement the functionality of processing circuitry 180 as described herein. Software 183 may be, or include, firmware.

FIG. 1 depicts system 100 in a use scenario in which it is monitoring oxygenation of tissue 120, which contains a protoporphyrin IX (PPIX) 122. FIG. 1 depicts spectral bands 140 of illumination and signals associated with system 100. These include a fluorescence excitation band 142 of PPIX 122 and a fluorescence spectral band 144.

Light source 110 is configured to illuminate tissue 120 with illumination 118. Illumination 118 has spectral components 117 within fluorescence excitation band 142, thereby generating one or both of a prompt fluorescence (PF) signal 124 and a delayed fluorescence (DF) signal 126 propagating from tissue 120. Each of signals 124 and 126 has spectral components within fluorescence spectral band 144. Photodetector 130 is configured to detect one or both of PF signal 124 and DF signal 126, respectively, as detected PF signal 134 and detected DF signal 136. Processing circuitry 180 is configured to generate, at least in part from detected DF signal 136, an oxygenation state 188 indicative of oxygenation of tissue 120. Examples of oxygenation state 188 include a value, or a range of values, of oxygen concentration or partial pressure in tissue 120. Other examples of oxygenation state 188 include hypoxic, not hypoxic, a degree of hypoxia, or a probability of being hypoxic.

System 100 may also include an emission filter 132, between tissue 120 and photodetector 130, that transmits fluorescence spectral band 144 while not transmitting (e.g., absorbing or reflecting) spectral components 117. For example, emission filter 132 may have a passband that includes fluorescence spectral band 144 that includes the DF spectral band while not including the spectral components 117.

In embodiments, system 100 includes a camera 160 and processing circuitry 180 includes an image processor 187. Camera 160 detects DF signal 126 as a DF image 166, and may also detect PF signal 124 as PF image 164, which may be stored in memory 182. In embodiments, processing circuitry 180 and image processor 187 are configured to generate the oxygenation state 188 at least in part from DF image 166. Image processor 187 may be part of processor 186.

Camera 160 may include photodetector 130, e.g., photodetector 130 may correspond to one or more sensor pixels of camera 160. In embodiments, system 100 includes a photodetector 190, which may be separate from one or both of photodetector 130 and camera 160. Photodetector 190 may include a fiber probe for capturing light emitted by tissue 120, and may be used for sensing oxyphor phosphorescence lifetime.

Memory 182 may store a lookup table 171 that maps DF signal intensities to respective tissue oxygenation levels. Examples of tissue oxygenation levels include a value, or range of values, of concentration or a partial pressure of oxygen in tissue 120. In embodiments, a DF signal intensity equal to zero or below a minimum value may correspond to well-oxygenated tissue, while a DF signal intensity above a threshold, e.g., one associated with necrotic tissue, may indicate full hypoxia.

In embodiments, light source 110 is a pulsed light source, such as a pulsed laser, and illumination 118 is a pulsed optical beam that includes excitation pulses 119. In such embodiments, photodetector 130 (and/or camera 160) may be gated and synchronized to light source 110 such that system 100 detects the DF signal 126 (and/or DF image 166) during a DF time window that does not overlap with any of light pulses of illumination 118. When illumination 118 is a pulsed optical beam having a temporal pulse spacing between excitation pulses 119, an exposure time of camera 160 may be set to be at least fifty times the temporal pulse spacing.

Photodetector 130 may be configured to detect PF signal 124 during a PF time window that either temporally overlaps an excitation pulse 119 and/or a time interval within 100 ns following an excitation pulse 119. In such embodiments, processing circuitry 180 may be configured to generate oxygenation state 188 from a ratio of detected DF signal 136 and detected PF signal 134. Photodetector 130 may have a blanking interval coincident with an excitation pulse 119. Processing circuitry 180 may include control circuitry 184, communicatively coupled to light source 110 and photodetector 130, that controls sequencing of excitation pulses 119 and time-gates photodetector 130. Control circuitry 184 may be, or include, a field-programmable gate array (FPGA), an application-specific integrated circuit (ASIC), a microcontroller, or any combination thereof.

Embodiments of system 100 include a radiation source 150, which is configured to apply a beam 158 of ionizing radiation to tissue 120. Radiation source 150 may be configured for ultra-high dose rate radiation therapy (e.g., ≥40 Gy/s.) or treatment of tissue 120. Examples of radiation source 150 include electron accelerators, such as a medical linear accelerator.

### 2. Method

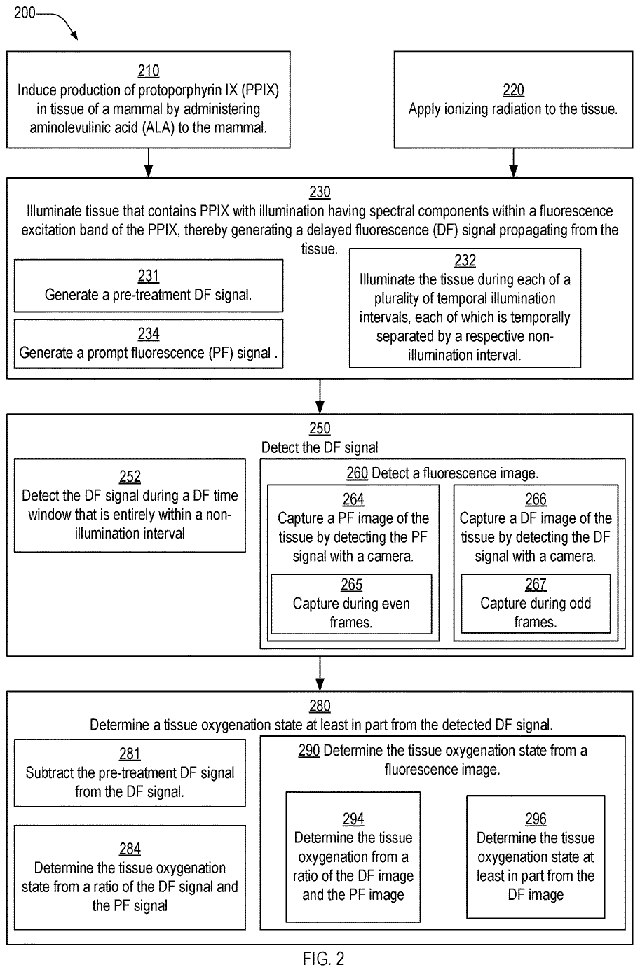

FIG. 2 is a flowchart illustrating a tissue-oxygenation monitoring method 200, which may be implemented by system 100. For example, method 200 may be implemented by processor 186 executing non-transitory computer-readable instructions of software 183. Method 200 includes at least one of steps 210, 220, 230, 250, 260, 280, and 290.

The following description of method 200 includes parenthetical numbers following terms recited by the method. The parenthetical number indicates that the element associated with the number in parentheses is an example of the term. For example, the description of step 264 below recites “detecting the PF signal with a camera (160),” which means that camera 160 of FIG. 1 is an example of the camera introduced in step 264.

Step 210 includes inducing production of PPIX (122) in tissue (120) of a mammal by administering aminolevulinic acid (ALA) to the mammal. Step 220 includes applying ionizing radiation (158) to the tissue, e.g., at a dose rate exceeding 40 Gy/s. Step 230 includes illuminating the tissue, which contains PPIX, with illumination (118) having spectral components within a fluorescence excitation band of the PPIX, thereby generating a delayed fluorescence (DF) signal (126) propagating from the tissue.

Step 230 may include one or more of steps 231, 232, and 234. Step 231 includes generating a pre-treatment DF signal, and may be performed prior to one or both of steps 210 and 220. Step 232 includes illuminating the tissue during each of a plurality of temporal illumination intervals, for example, when the illumination is a pulsed optical beam. Each pair of temporal illumination intervals is temporally separated by a respective non-illumination interval. Step 234 includes generating a prompt fluorescence (PF) signal (124).

Step 250 includes detecting the DF signal. When method 200 includes step 232, it may also include step 252, which includes detecting the DF signal during a DF time window that is entirely within a non-illumination interval.

Step 250 may include step 260, which includes detecting a fluorescence image. Step 260 includes one or both of steps 264 and 266. For example, when step 230 includes step 234, step 260 may include step 264. Step 264 includes capturing a PF image of the tissue by detecting the PF signal with a camera (160). Step 264 may include step 265, which includes capturing the PF image during even frames of the camera. Step 266 includes capturing a DF image (166) of the tissue by detecting the DF signal with the camera. Step 266 may include step 267, which includes capturing the DF image during odd frames of the camera.

Step 280 includes determining a tissue oxygenation state (188) at least in part from the detected DF signal. Step 280 may include determining, from an intensity of the DF signal, the tissue oxygenation state (188). Step 280 may include comparing an intensity of the DF signal to a plurality of reference DF signal intensities, each mapped to a tissue oxygenation level. The DF signal intensities and oxygenation levels may be stored in lookup table 171.

When step 230 includes step 231, step 280 may include step 281. Step 281 includes determining the tissue oxygenation state at least in part by subtracting the pre-treatment DF signal from the DF signal. Step 280 may include a step 284, e.g., when step 230 includes step 234. Step 284 includes determining the tissue oxygenation state from a ratio of the DF signal and the PF signal.

When step 260 includes step 264, step 280 may include step 294; and when step 260 includes step 266, step 280 may include step 296. Step 294 includes determining the tissue oxygenation from a ratio of the DF image and the PF image. Step 296 includes determining the tissue oxygenation state at least in part from the DF image.

### 3. Significance

Radiation Therapy (RT) is the most common non-surgical method for cancer treatment[1,2], used for curative purposes as adjuvant therapy after surgery in approximately 50% of cases[3-6]. The goal of RT is to deliver the toxic dose to the tumor tissues while also giving sufficiently low dose to the limiting healthy tissues that cannot be avoided[7,8], while assigning treatment margins to account for uncertainties related to tumor motion, volume definition[9-11], and the limitations of conformity[12,13]. These factors inevitably result in some exposure of healthy tissues and organs around the tumor as the beams enter and exit the body. This undesired radiation dose deposition increases the risk of treatment morbidity such as inflammation, desquamation, fibrosis or secondary malignancy occurrence post treatment, ultimately increasing healthcare costs, or eventual disease related mortality[14-16].

Newer delivery techniques are constantly being investigated to reduce damage to this surrounding normal tissue. Recent studies have shown that ultra-high dose rate RT (UHDR-RT) (>40 Gy/s) results in a significant reduction of radiation damage to normal tissue[17], although the mechanisms of action are still not well understood at this point. In conventional RT (CONV-RT), the mean dose rates are approximately 0.03 Gy/s, [18]. During UHDR-RT rapid oxygen transients have been observed [19,20], although the magnitude of them has been the subject of substantial speculation and hypothesis, because of how dominant oxygen is in influencing radiation damage. The most dominant known pathway of damage is through the widespread production of reactive oxygen species (ROS).

The exact mechanism behind UHDR-RT, or the so-called FLASH effect, is still subject to significant research with ongoing debate about the cause. Several radiobiological hypotheses have been proposed [21], but none have been conclusively proven. Most of the proposed mechanisms are linked to oxygen consumption to the point of complete depletion in tissues [21], however recent in vivo studies have demonstrated that complete depletion does not happen in vivo, and solution data indicates that conversely UHDR-RT actually reduces the amount of oxygen consumed per unit dose, as compared to CONV-RT [22]. This fact appears under-appreciated in interpretation of the FLASH effect.

According to the oxygen fixation hypothesis, DNA damage caused by free radicals is made more permanent by the presence of molecular oxygen, through peroxyl radical formation [23], and measuring the process of oxygen being consumed in the ROS formation might be considered as a surrogate for peroxyl radical formation since this is where the oxygen goes when it is depleted. This phenomenon may be related to the oxygen enhancement ratio (OER), because the presence of oxygen and the formation of peroxyl radicals in DNA are thought to increase cell death by up to a factor of 3 times as compared to hypoxic conditions [24,25]. Very interestingly, UHDR-RT induced oxygen transients are observable in vivo, whereas CONV-RT oxygen consumption is not, because of how slow the depletion is occurring. However, quantifying the oxygen changes in vivo is complex because of how heterogeneous and fast the changes are, combined with the fact that every measurement system really only provides partial information about the oxygen.

The gold standard for oxygen measurement, the polarographic electrode, can provide precise partial pressure (pO₂) readings, but they are invasive and allow only slow, limited point measurements [26-28] which can even distort the local oxygen environment itself from the mechanical pressure it induces. Alternatively, oxygen measurements relying on triplet state fluorescence quenching offer a contactless, wide-field, and fast option for monitoring oxygen.

Oxyphor is a dendritic phosphorescent probe with different metallo-tetra-aryl-phthalimido-porphyrins as a core chromophore [29]. PdG4 Oxyphor is based upon palladium as the inner atom, and has a quantum yield greater than 23% making it an exceptionally bright phosphorescent sensor. PdG4 was designed as a ~7 nm diameter molecule to specifically sense extracellular environments.

So, oxygen measurement with this implicitly is a mixture of interstitial fluid (ISF) and blood plasma. For this reason, measuring oxygen using PdG4 only provides information on extracellular oxygen fraction of tissues. Cao et al.[19] utilized pO₂ measurements based on PdG4 lifetime to demonstrate that UHDR-RT resulted in approximately twice as much oxygen depletion in normal tissue compared to tumor tissue. El Khatib et al. [20] utilized this to demonstrate similar consumption in vivo with proton UHDR-RT and that the consumption rate depended upon the initial pO₂ of the tissue, indicating that consumption in tumor tissue at low pO₂ was less than that in higher pO₂ normal tissues. No oxygen decrease has been documented during CONV-RT because of the comparatively faster resupply from diffusion from the supplying capillary network in tissue.

Conclusions from these studies show that UHDR-RT 02 consumption in vivo was in the range of 0.1-0.2 mmHg/Gy (0.14-0.28 μM/Gy) range depending upon the tissue and conditions [19], but this necessarily needs to be defined as a factor of the initial pO₂ as well [30] for an approximate consumption rate of 0.0083 mmHg/Gg per initial mmHg. But clearly one remaining controversial part of this is the site of measurement and how to interpret these rate changes with relevance to the oxygen fixation damage of DNA, within the cellular nucleus.

Oxygen diffuses from the blood to cells through the capillary network with oxygen levels averaging 95 mmHg and 40 mmHg in capillaries and interstitial fluid, respectively [31]. Mitochondria consume oxygen and require at least 5 mmHg [32] to function properly, creating a decreasing gradient of oxygen between the ISF and cell nuclei. Cells can have varying numbers of mitochondria, ranging from basically none to as many as 2000 or more per cell [33], depending on the tissue type.

Due to the proximity of these mitochondria to the cell's nucleus, PpIX is synthesized close to the nucleus, making it an ideal reporter of oxygen activity at the DNA level. The oxygen reporting capability using DF from PpIX has been reported in many studies [34-37]. The PpIX is endogenously created as a step in the hemoglobin biosynthesis pathway, within the mitochondria inside cells. Since DNA damage is the primary factor leading to cell death during radiotherapy, PpIX can serve as a unique tool for fast measurement of oxygen depletion during UHDR-RT, providing more relevant information about oxygen levels nearly the intracellular region of interest.

### 4. Methods

### 4.1. Optical Probes

Two optical probes were used to assess oxygen dynamics in vivo during UHDR irradiation, including exogenously injected Oxyphor PdG4 and endogenously produced PpIX form administration of aminolevulinic acid (ALA). These are both oxygen sensitive luminescent molecules relying on triplet quenching by molecular oxygen, but implicitly sensing different tissue compartments. However, the major benefit of both sensors is that they are minimally perturbative to the in vivo tissue and both may be used in living animal tissue, and PpIX can be readily translated into human tissue measurement.

Using a fast APD detector, the phosphorescence time decay of PdG4 was registered at a sampling rate of 2 Hz and used to recover the phosphorescent lifetime of the probe, linked to ambient oxygen levels by the Stern-Volmer equation [38]. While this method provides a fast and robust way to measure oxygen, it does not allow for spatial resolution of the oxygen distribution in tissue. Using the imaging system described here [37], PpIX DF was also recorded at a frame rate of 5 frames per second (fps).

The PpIX fluorescence images were captured using a single-photon sensitive intensified CMOS camera synchronized with a 50-mW average output power 635-nm modulated diode laser. The laser operated with 20 μs pulses at a repetition rate of 500 Hz and was partially collimated to irradiate an 8 cm diameter area, resulting in a temporally averaged irradiance of 500 μW/cm2 at the sample. Irradiance measurements were obtained using a power meter coupled with a photodiode power sensor.

The camera's intensifier was gated using a field-programmable gate array (FPGA) which was synchronized with the laser pulses. This FPGA performed custom pulse sequencing and control of the image acquisition timing. For prompt fluorescence (PF) and DF pulses, different pulse timings were utilized. PF employed a 100 ns pulse width with no delay relative to the laser pulse, while DF had a pulse width of 1975 μs and a delay of 2 μs after the laser pulse. The exposure time was set to a 100-ms time window, which integrated 50 laser pulse gates in each image frame. The FPGA switched between PF and DF acquisition for even and odd frames, respectively. This sequential acquisition of PF and DF of PpIX allowed for an effective frame rate of 10 fps, facilitating real-time reconstruction of the normalized hypoxia image (i.e., DF/PF).

In the subsequent sections of this disclosure, the PpIX signal is denoted by the variable R=DF/PF. This choice of notation is intended to normalize the DF signal, taking into consideration the tissue's optical properties and PpIX concentration. By employing this normalization, may mitigate the impact of these factors and enhance the accuracy and comparability of the measurements. The PpIX fluorescent signal passed through a 697±37 nm, OD 6 band-pass filter to remove any remaining laser emission that could interfere with the detection spectral window. All images were acquired with 2×2 pixel binning, resulting in a final image size of 800×600 pixels.

PdG4 has two main excitation bands at 448 nm and 637 nm and phosphoresces near 813 nm, which is within the near-infrared window of tissue. PpIX's main excitation peak is at 405 nm, but it also has a series of Q bands, including one at 635 nm, and emits broadband with two primary peaks at 635 and 700 nm. In this work, both probes were excited with the same laser at 635 nm, to allow good penetration of light into tissue, and emission was detected beyond 650 nm for PpIX and beyond 750 nm for PdG4. Both devices were synchronized to allow for parallel acquisition of the PpIX and PdG4 signals. The PdG4 signal was acquired using a multimodal fiber optic probe coupled to the APD detector and positioned near the tissue surface. It is important to note that because of significant spectral cross talk between the two types of measurement, that co-located sensing of the signals was not possible, and so this study was carried out with paired measurements of the two signals on opposites sides of the same animal, during all functional studies.

### 4.2. Animal Preparation

All procedures followed the protocol approved by the Dartmouth Institutional Animal Care and Use Committee. Nude female mice 6-8 weeks of age (Charles River Labs, Wilmington, MA) were purchased for use in this study. The mice were housed in the Dartmouth vivarium and fed special low fluorescence diet (MP biomedical purified diet). On the day of imaging, mice were anesthetized, and ALA (Sigma-Aldrich, MO) dissolved in phosphate-buffered saline (PBS) (1×, Corning, NY) was injected intraperitoneally (250 mg/kg) three hours prior to imaging. At 10 to 30 minutes prior to imaging, a 50 μL of a solution of PdG4 at 10 μM concentration, was dissolved in PBS was injected into the skin of the animal at the site to be measured.

For imaging, mice were anesthetized using isoflurane (Fluriso, Vet One, ID) and 1 L/min oxygen flow. The amount of isoflurane varied between 1% and 3% to regulate the physiological oxygen baseline.

### 4.3. UHDR Delivery & Dose Quantification

A clinical linear accelerator (LINAC) (Trilogy, Varian, CA) was converted to deliver UHDR electron beams, using an experimental mode that retracted the x-ray target and flattening filter from the electron beam's path in a routine manner developed by Rahman et al. [39]. The prescribed dose was controlled by selecting the number of delivered pulses using a calibrated dose control system described in the work by Ashraf et al. [40]. The actual delivered dose was monitored using radiochromic film dosimeters (EBT-XD, Ashland, Bridgewater, NJ) [41] placed underneath the animal, for each irradiation. Film readout was quantified by digitizing using a flatbed scanner (Epson, Suwa, Japan) at a resolution of 96 dots per inch with a 48-bit depth (16 bits per color channel) and converted to dose by calibration measurements, as completed with the same Linac.

The calibration curve relating the change in optical density to dose was obtained at conventional dose rates for a 9 MeV beam by delivering known doses ranging from 0 to 6000 centigray to six different pieces of film [39]. Each film was from a single batch and found to have a variation of less than 1.4% across repeated deliveries. Minor dose reduction was seen in the first 4 to 6 pulses before the output stabilized, after which its stability was within 3%, and this dose rate reduction was consistent for all samples. Thus, about 97% of the dose delivered was at UHDR values for these experiments, and sample-to-sample variation in instantaneous and mean dose rate was estimated to be less than 3%.

Additionally, optical stimulated luminescent dosimeters (OSLDs) were calibrated and irradiated with the beam in UHDR mode to validate film dose measurements as part of this study [39]. The OSLDs were placed along with film on the central axis of a broad, open-field UHDR beam. The surface dose was measured with both dosimeters from delivery of 15 pulses 5 times at the isocenter. The dose recorded from the film for each treatment delivery of 15 pulses agreed with the OSLD dose to within 3%, suggesting the film could be used to characterize the beam accurately.

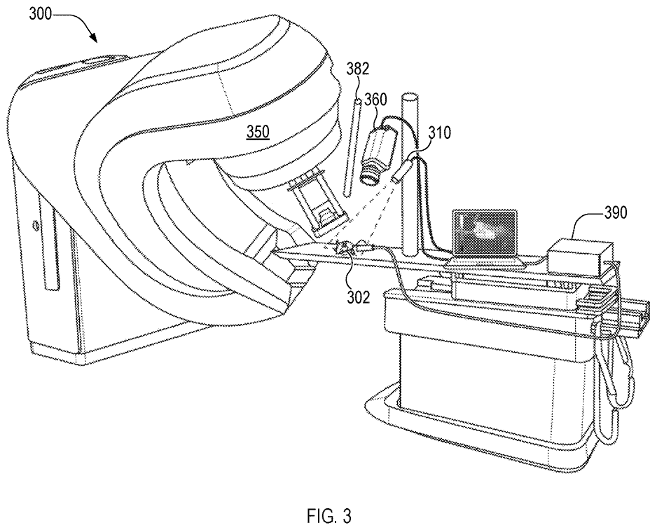

FIG. 3 is a schematic of a tissue-oxygenation monitoring system 300, which is an example of tissue-oxygenation monitoring system 100. System 300 includes a laser 310, a photodetector 130, a medical linear accelerator 350, a camera 360, and a fiber-coupled detector probe 390, which are respective examples of light source 110, photodetector 130, radiation source 150, camera 160, and photodetector 190.

In an example use scenario, medical linear accelerator 350 irradiates a mouse 302 as it is imaged by both systems, camera 360 imaging for PpIX delayed fluorescence (intracellular pO₂) and fiber-coupled detector probe 390 sensing oxyphor phosphorescence lifetime (extracellular pO₂). Mice were exposed to varying doses of radiation, specifically 8.4 Gy, 11.2 Gy, 22.4 Gy, and 28 Gy.

These doses corresponded to 3-4, 8, and 10 pulses, respectively. The low-dose prescription of 3 pulses occasionally resulted in 4 pulses due to signal delay in the Linac. To address this variability consistently throughout this disclosure, radiation doses corresponding to 3-4 pulses (specifically, 8.4 Gy-11.2 Gy) are designated as “10 Gy irradiations”. This allowed standardization of the reported doses and facilitates clarity and comparability in findings across dose levels. Each 10 MeV UHDR electron beam delivered was 10×10 cm² which corresponded to total body irradiation. After irradiation, the mice were euthanized and imaged again to establish the signal post-euthanasia as a reference for total hypoxia.

To reduce electronic noise and charge accumulation that resulted from direct interaction of camera 360 with high-energy scattered electrons, the PpIX fluorescence imaging system was shielded using a 5 mm thick lead sheet 382 wrapped around camera 360.

### 4.4. Data Processing

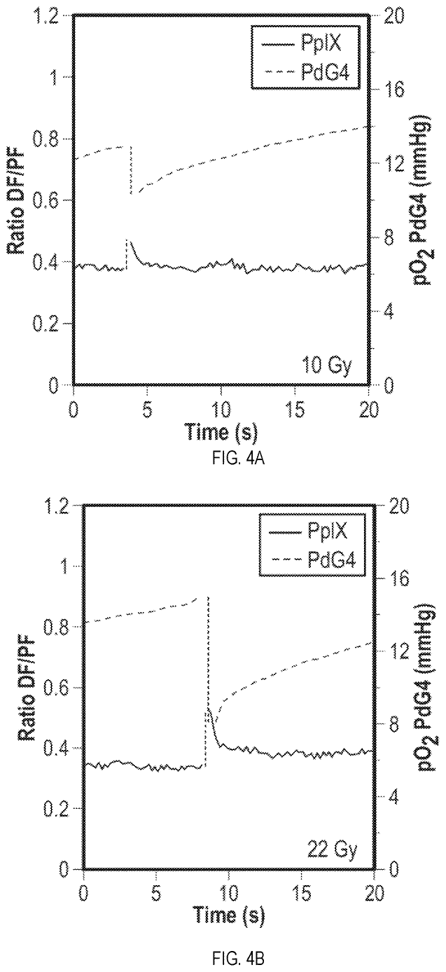

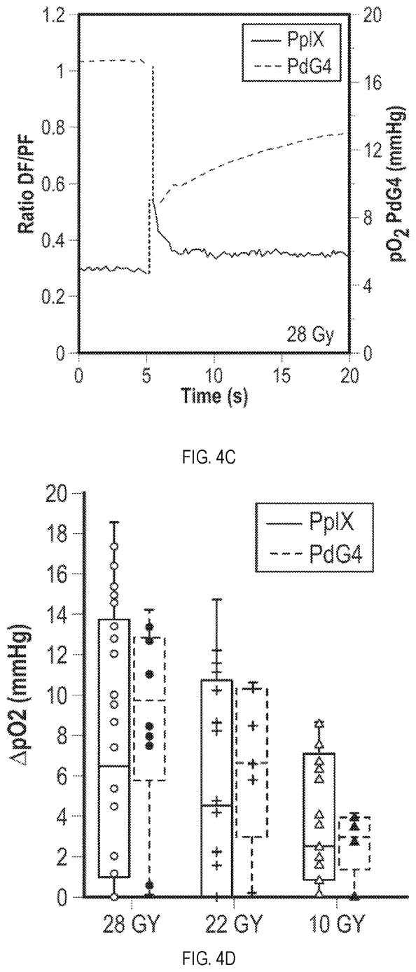

PpIX ratiometric signal time profiles were generated from regions of interest (ROIs), including 30×30 pixels, which were selected to correspond to areas of mouse 302, located either on the leg or the flank of the animal. PpIX ratiometric signal time profiles were generated from the average pixel value of the ROIs over time. To eliminate background noise, each time profile was background-subtracted. To exclude saturated PpIX signals resulting from the occurrence of the UHDR beam, saturated data were excluded from all data displayed in this study, resulting in the discontinuous curves in FIGS. 4A-4D. To highlight the dependency of oxygen depletion on initial oxygen content of the tissue, data were fitted using a model derived from the Michaelis equation:

$$Δ p O 2 = \frac{ΔpO2\_max \times p O 2}{K + p O 2} \qquad ( 1 )$$

where ΔpO2 is the amount of oxygen depleted during UHDR irradiation, ΔpO2_max is the maximum oxygen depletion for a given dose, pO2 is the initial tissue oxygenation (or surrogate measurement of pO₂ in the case of PpIX ratiometric signal) prior to irradiation and K is a constant reflecting the rate of oxygen consumption. In the same way, the relationship between PpIX ratiometric measurements and PdG4 based measurements were fitted using a linear model. All models were fitted using an iterative non-linear least square method (Matlab R2018a, The MathWorks Inc., Natick, MA).

### 5. Results

### 5.1. Measurement of the Oxygen Transient During UHDR-RT

FIGS. 4A-4D depict the outcomes of UHDR irradiation of mice with various doses. FIGS. 4A, 4B, and 4C display the temporal profiles of oxygen measurement employing PdG4 (dashed line) and PpIX (solid line) when irradiated with 10 Gy, 22 Gy, and 28 Gy, respectively. In each graph, the discontinuity in the curve corresponds to beam delivery. The PdG4 measurement shows a slightly longer discontinuity than the PpIX measurement due to the slower sampling rate of the former. As the beam was delivered, there was an immediate reduction in oxygen levels, which was reflected in an increase in the PpIX ratiometric signal. The extent of this oxygen depletion is seen to increase with the dose. The initial pO₂ values also align between the PdG4 and PpIX measurements.

FIG. 4D presents the overall results from all irradiations, where both PdG4 and PpIX based oxygen depletion (ΔpO₂) measurements are expressed in mmHg. While PdG4 lifetime measurement provides an absolute measurement of pO₂, PpIX ratiometric signal was calibrated using the PdG4 based O₂ measurement as described by Petusseau et al [42]. The range of PpIX-based ΔpO₂ is observed to be greater than that of the PdG4 measurement for each dose. However, both measurements follow the same trend and indicate that ΔpO₂ decreases with the delivered dose. In this study, each mouse was irradiated once or twice depending on the total dose, and PdG4 phosphorescence was collected from the left leg, while PpIX ratiometric signal was acquired from the right leg, upper back, and lower back of the mice, resulting in a higher number of data points presented for PpIX ratiometric measurements.

### 5.2. Dependance of Oxygen Depletion on Initial Oxygen Levels

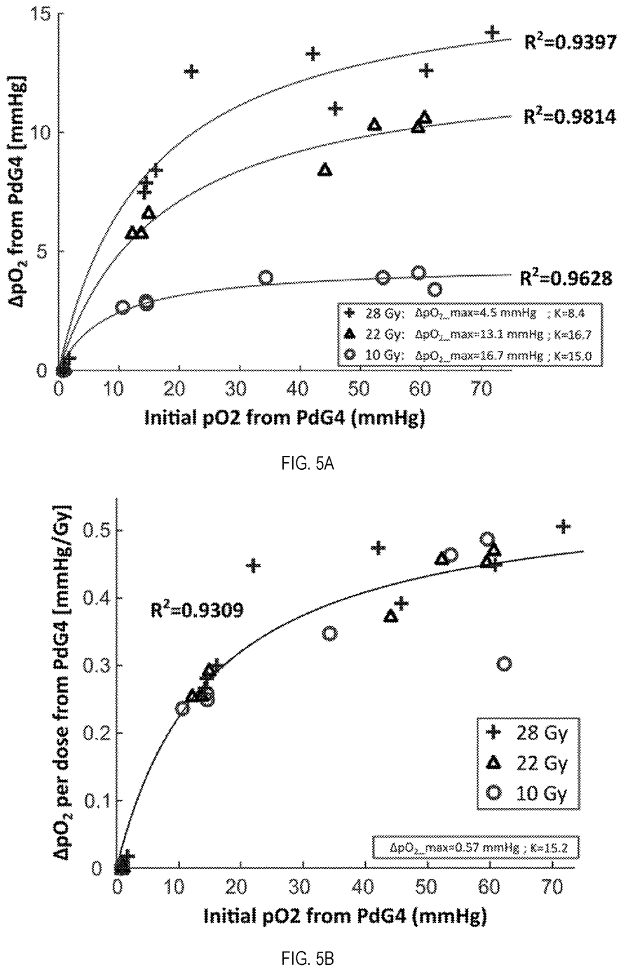

As the extent of oxygen depletion appeared to vary considerably based on the results obtained from FIGS. 4A-4D, FIGS. 5A and 5B show the results of investigating the relationship between ΔpO₂ and the initial tissue pO₂, as measured using PdG4. For each delivered dose, oxygen depletion increases with the initial pO₂ and eventually saturates at higher initial oxygen levels.

FIG. 5A illustrations the relationship between tissue initial pO₂ and UHDR induced oxygen depletion, as measured using PdG4 optical signal. Each dataset corresponding to varying delivered doses is fitted using equation (1). FIG. 5B shows pO₂ depletion during UHDR, normalized to dose. The data points corresponding to each delivered dose were fitted using the model described in section 4.4. This model closely fits the data and demonstrates that saturation is reached at lower initial pO₂ levels for lower delivered doses.

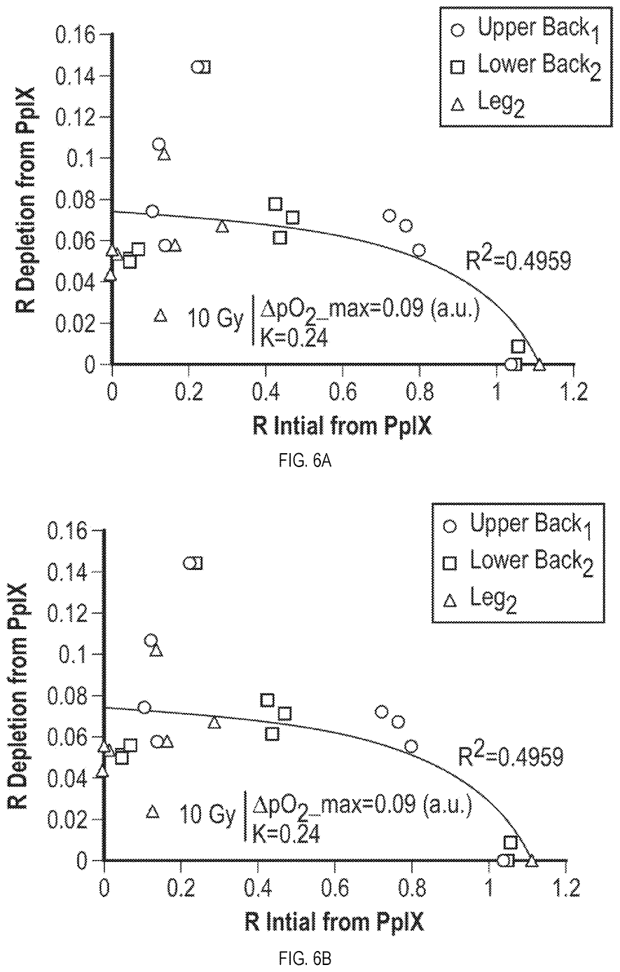

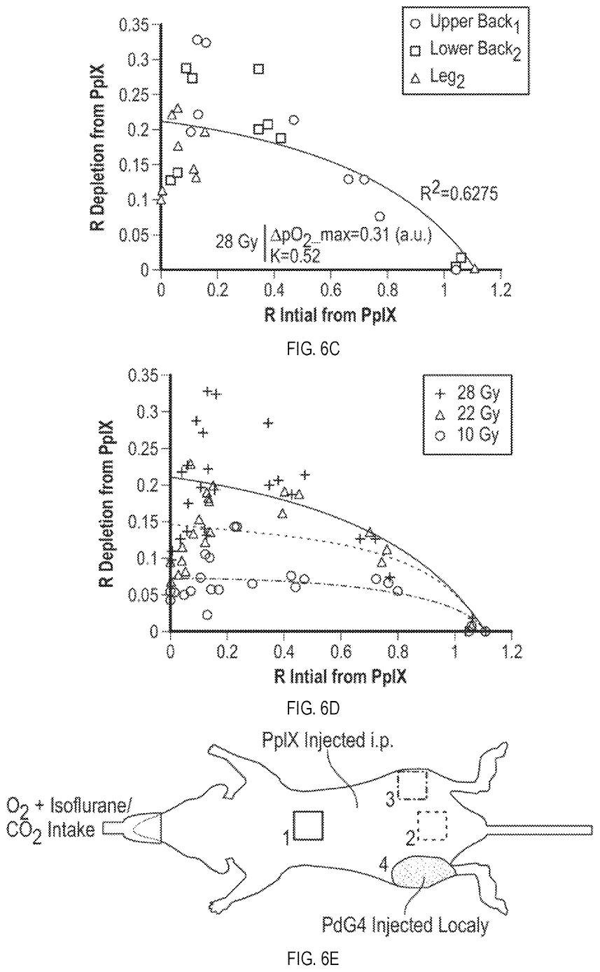

FIGS. 6A-6E show the dependence of ΔpO₂ on the initial pO₂ values, as measured using the PpIX ratiometric signal. FIGS. 6A-6D show a relationship between tissue initial R value and UHDR-induced R depletion (ΔR), as measured using PpIX optical signal. Each dataset corresponds to varying delivered doses is fitted using equation (1).

FIGS. 6A through 6C depict this relationship for varying delivered UHDR doses, corresponding to those used in FIGS. 5A and 5B. Each plot represents measurements from a different area of the mouse body, i.e., the right leg, upper back, or lower back, and is represented by a different shape.

The ROIs used for this purpose are described in FIG. 6E and numbered accordingly. FIG. 6E shows a distribution of each optical probe in the murine model and location of the ROIs used for data collection. The shaded area corresponds to the region of PdG4 injection used in the data from FIGS. 5A and 5B. Each dataset corresponding to a different UHDR dose was fitted using the model described in section 4.4. In the case of PpIX based measurements, lower R-squared values were indicative of spatial heterogeneities in O₂ depletion across the bodies of the specimens. FIG. 6D summarizes the previous plots to facilitate comparison and shows fitting in accordance with the delivered dose.

### 5.3. Relationship Between Intra and Extracellular Oxygen Depletion

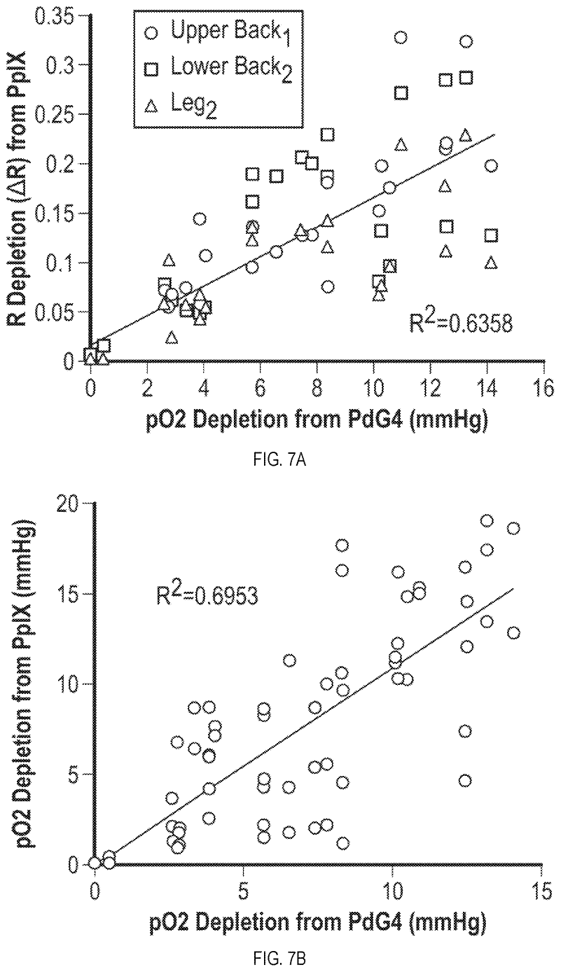

FIGS. 7A and 7B illustrate the relationship between oxygen depletion measured using both PdG4 and PpIX probes. FIG. 7A graphically compares oxygen depletion during varying doses of UHDR irradiation, as measured with PpIX (ΔR) and PdG4 (ΔpO₂) probes. In FIG. 7A, the depletion based on the ratiometric signal (ΔR) is plotted against the PdG4 signal (ΔpO₂). Each datapoint corresponds to one of the three ROIs described in FIG. 6E and is represented by a different shape on the graph. Each point is correlated to the corresponding PdG4 measurement, which explains the vertical alignment of the data points on the graph. In FIG. 7B, the same data is displayed with correction applied to ΔR to express pO₂ measurements in mmHg. That is, FIG. 7B is a comparison of the corrected data ΔR from FIG. 7A, with the absolute pO2 measurement provided by the PdG4 phosphorescence lifetime.

### 6. References

1. Mayles P, Nahum A, Rosenwald J C eds. Handbook of Radiotherapy Physics: Theory and Practice. Boca Raton: CRC Press, 2007.
2. Citrin D E. Recent Developments in Radiotherapy. New England Journal of Medicine 2017; 377:1065-75.
3. Atun R, Jaffray D A, Barton M B et al. Expanding global access to radiotherapy. The Lancet Oncology 2015; 16:1153-86.
4. Adjuvant radiotherapy for rectal cancer: a systematic overview of 8507 patients from 22 randomised trials. The Lancet 2001; 358:1291-304.
5. Kong A, Johnson N, Kitchener H C et al. Adjuvant radiotherapy for stage I endometrial cancer. Cochrane Database of Systematic Reviews 2012, DOI: 10.1002/14651858.CD003916.pub3.
6. Cancer Treatment Reports. U.S. Department of Health, Education, and Welfare, Public Health Service, National Institutes of Health, 1987.
7. Formenti S C, Demaria S. Systemic effects of local radiotherapy. The Lancet Oncology 2009; 10:718-26.
8. De Ruysscher D, Niedermann G, Burnet N G et al. Radiotherapy toxicity. Nat Rev Dis Primers 2019; 5:1-20.
9. Korreman S S. Motion in radiotherapy: photon therapy. Phys Med Biol 2012; 57: R161.
10. Ozhasoglu C, Murphy M J. Issues in respiratory motion compensation during external-beam radiotherapy. International Journal of Radiation Oncology—Biology—Physics 2002; 52:1389-99.
11. Berbeco R I, Nishioka S, Shirato H et al. Residual motion of lung tumours in gated radiotherapy with external respiratory surrogates. Phys Med Biol 2005; 50:3655.
12. Feuvret L, Noël G, Mazeron J-J et al. Conformity index: A review. International Journal of Radiation Oncology—Biology—Physics 2006; 64:333-42.
13. Baumert B G, Lomax A J, Miltchev V et al. A comparison of dose distributions of proton and photon beams in stereotactic conformal radiotherapy of brain lesions. International Journal of Radiation Oncology—Biology—Physics 2001; 49:1439-49.
14. Effects of radiotherapy and of differences in the extent of surgery for early breast cancer on local recurrence and 15-year survival: an overview of the randomised trials. The Lancet 2005; 366:2087-106.
15. Green J A, Kirwan J M, Tierney J F et al. Survival and recurrence after concomitant chemotherapy and radiotherapy for cancer of the uterine cervix: a systematic review and meta-analysis. The Lancet 2001; 358:781-6.
16. Bartelink H, Horiot J-C, Poortmans P et al. Recurrence Rates after Treatment of Breast Cancer with Standard Radiotherapy with or without Additional Radiation. New England Journal of Medicine 2001; 345:1378-87.
17. Favaudon V, Caplier L, Monceau V et al. Ultrahigh dose-rate FLASH irradiation increases the differential response between normal and tumor tissue in mice. Sci Transl Med 2014; 6: 245ra93.
18. Ashraf M R, Rahman M, Zhang R et al. Dosimetry for FLASH Radiotherapy: A Review of Tools and the Role of Radioluminescence and Cherenkov Emission. Frontiers in Physics 2020; 8.
19. Cao X, Zhang R, Esipova T V et al. Quantification of Oxygen Depletion During FLASH Irradiation In Vitro and In Vivo. International Journal of Radiation Oncology—Biology—Physics 2021; 111:240-8.
20. El Khatib M, Van Slyke A L, Velalopoulou A et al. Ultrafast Tracking of Oxygen Dynamics During Proton FLASH. International Journal of Radiation Oncology—Biology—Physics 2022; 113:624-34.
21. Hughes J R, Parsons J L. FLASH Radiotherapy: Current Knowledge and Future Insights Using Proton-Beam Therapy. IJMS 2020; 21:6492.
22. Sunnerberg J P, Zhang R, Gladstone D et al. Mean dose rate in ultra-high dose rate electron irradiation is a significant predictor for O₂ consumption and H₂H₂ yield. Phys Med Biol 2023, DOI: 10.1088/1361-6560/ace877.
23. Grimes D R, Partridge M. A mechanistic investigation of the oxygen fixation hypothesis and oxygen enhancement ratio. Biomed Phys Eng Express 2015; 1:045209.
24. Bristow R G, Hill R P. Hypoxia, DNA repair and genetic instability. Nat Rev Cancer 2008; 8:180-92.
25. Antonovic L, Lindblom E, Dasu A et al. Clinical oxygen enhancement ratio of tumors in carbon ion radiotherapy: the influence of local oxygenation changes. J Radiat Res 2014; 55:902-11.
26. Vaupel P, Schlenger K, Knoop C et al. Oxygenation of Human Tumors: Evaluation of Tissue Oxygen Distribution in Breast Cancers by Computerized O2 Tension Measurements 1. Cancer Research 1991; 51:3316-22.
27. Swartz H M, Flood A B, Schaner P E et al. How best to interpret measures of levels of oxygen in tissues to make them effective clinical tools for care of patients with cancer and other oxygen-dependent pathologies. Physiological Reports 2020; 8: e14541.
28. Höckel M, Schlenger K, Mitze M et al. Hypoxia and radiation response in human tumors. Seminars in Radiation Oncology 1996; 6:3-9.
29. Esipova T V, Barrett M J P, Erlebach E et al. Oxyphor 2P: A High-Performance Probe for Deep-Tissue Longitudinal Oxygen Imaging. Cell Metabolism 2019; 29:736-744.e7.
30. Van Slyke A L, El Khatib M, Velalopoulou A et al. Oxygen Monitoring in Model Solutions and In Vivo in Mice During Proton Irradiation at Conventional and FLASH Dose Rates. Radiat Res 2022; 198:181-9.
31. Feher J. 6.1—The Mechanics of Breathing. In: Feher J (ed.). Quantitative Human Physiology (Second Edition). Boston: Academic Press, 2017, 623-32.
32. Gnaiger E. Oxygen Conformance of Cellular Respiration. In: Roach R C, Wagner P D, Hackett P H (eds.). Hypoxia. Boston, MA: Springer U S, 2003, 39-55.
33. Alberts B, Johnson A, Lewis J et al. Molecular Biology of the Cell. 4th ed. Garland Science, 2002.
34. Mik E G, Stap J, Sinaasappel M et al. Mitochondrial PO2 measured by delayed fluorescence of endogenous protoporphyrin IX. Nat Methods 2006; 3:939-45.
35. Mik E G, Johannes T, Zuurbier C J et al. In Vivo Mitochondrial Oxygen Tension Measured by a Delayed Fluorescence Lifetime Technique. Biophysical Journal 2008; 95:3977-90.
36. Mik E G. Measuring mitochondrial oxygen tension: From basic principles to application in humans. Anesthesia and Analgesia 2013; 117:834-46.
37. Pétusseau A, Bruza P, Pogue B. Protoporphyrin IX delayed fluorescence imaging: a modality for hypoxia-based surgical guidance. J Biomed Opt 2022; 27:106005.
38. Lo L-W, Vinogradov S A, Koch C J et al. A New, Water Soluble, Phosphor for Oxygen Measurements in Vivo. In: Harrison D K, Delpy D T (eds.). Oxygen Transport to Tissue XIX. Boston, MA: Springer U.S. Pat. No. 1,997,651-6.
39. Rahman M, Ashraf M R, Zhang R et al. Electron FLASH Delivery at Treatment Room Isocenter for Efficient Reversible Conversion of a Clinical LINAC. International Journal of Radiation Oncology—Biology—Physics 2021; 110:872-82.
40. Ashraf M R, Rahman M, Cao X et al. Individual pulse monitoring and dose control system for pre-clinical implementation of FLASH-RT. Phys Med Biol 2022; 67:095003.
41. Jaccard M, Petersson K, Buchillier T et al. High dose-per-pulse electron beam dosimetry: Usability and dose-rate independence of EBT3 Gafchromic films. Med Phys 2017; 44:725-35.
42. Petusseau A F, Ochoa M, Reed M et al. Intracellular oxygen dynamics imaging for surgical guidance: introducing Pressure Enhanced Sensing Surgery (PRESS). In review.
43. Wilson D F, Cerniglia G J. Localization of tumors and evaluation of their state of oxygenation by phosphorescence imaging. Cancer Res 1992; 52:3988-93.

### 7. Combinations of Features

Features described above, as well as those claimed below, may be combined in various ways without departing from the scope hereof. The following enumerated examples illustrate some possible, non-limiting combinations.

(A1) A tissue-oxygenation monitoring method includes illuminating tissue that contains protoporphyrin IX (PPIX) with an illumination having spectral components within a fluorescence excitation band of the PPIX, thereby generating a delayed fluorescence (DF) signal propagating from the tissue. The method further includes detecting the DF signal and determining a tissue oxygenation state at least in part from the detected DF signal.

(A2) In embodiments of (A1), determining the tissue oxygenation state includes determining, from an intensity of the DF signal, the tissue oxygenation state as either hypoxic, not hypoxic, a degree of hypoxia, or a probability of being hypoxic.

(A3) In either of embodiments (A1) or (A2), determining the tissue oxygenation state includes comparing an intensity of the DF signal to a plurality of reference DF signal intensities, each mapped to a tissue oxygenation level.

(A4) In any of embodiments (A1)-(A3), illuminating the tissue further generates a prompt fluorescence (PF) signal, and determining the tissue oxygenation state includes determining the tissue oxygenation state from a ratio of the DF signal and the PF signal.

(A5) In any of embodiments (A1)-(A4), the tissue is of a mammal, and the method further includes inducing production of PPIX in the tissue by administering aminolevulinic acid (ALA) to the mammal.

(A6) In any of embodiments (A1)-(A5), the illumination is a pulsed optical beam, and illuminating the tissue includes illuminating the tissue during each of a plurality of temporal illumination intervals.

(A7) In embodiments of (A6), each pair of temporal illumination intervals is temporally separated by a respective non-illumination interval, and the method further includes detecting the DF signal during a DF time window that is entirely within a non-illumination interval.

(A8) In any of embodiments (A1)-(A7), detecting the DF signal includes capturing a DF image of the tissue by detecting the DF signal with a camera, and determining the tissue oxygenation state includes determining the tissue oxygenation state at least in part from the DF image.

(A9) In embodiments of (A8), illuminating the tissue further generates a prompt fluorescence (PF) signal, detecting includes capturing a PF image of the tissue by detecting the PF signal with the camera, and determining the tissue oxygenation state includes determining the tissue oxygenation from a ratio of the DF image and the PF image.

(A10) In embodiments of (A9), capturing the PF image includes capturing the PF image during even frames of the camera, and capturing the DF image includes capturing the DF image during odd frames of the camera.

(A11) In any of embodiments (A1)-(A10), the method further includes applying ionizing radiation to the tissue.

(A12) In embodiments of (A11), illuminating the tissue includes generating, prior to applying the ionizing radiation to the tissue, a pre-treatment DF signal, and determining the tissue oxygenation state includes determining the tissue oxygenation state at least in part by subtracting the pre-treatment DF signal from the DF signal.

(B1) A tissue-oxygenation monitoring system includes a light source configured to illuminate tissue with illumination having spectral components within a fluorescence excitation band of protoporphyrin IX (PPIX), thereby generating a delayed fluorescence (DF) signal propagating from the tissue. The system further includes a photodetector configured to detect the DF signal and processing circuitry communicatively coupled to the photodetector and configured to determine, at least in part from the detected DF signal, an oxygenation state indicative of oxygenation of the tissue.

(B2) In embodiments of (B1), the photodetector is a camera that detects the DF signal as a DF image, the processing circuitry includes an image processor, and the processing circuitry and the image processor are configured to generate the oxygenation state at least in part from the DF image.

(B3) In either of embodiments (B1) or (B2), the light source is a pulsed light source and the illumination is a pulsed optical beam.

(B4) In embodiments of (B3), the photodetector is gated and synchronized to the pulsed light source and is configured to detect the DF signal during a DF time window that does not overlap with any of light pulses of the pulsed optical beam.

(B5) In embodiments of (B4), the photodetector is further configured to detect a prompt fluorescence (PF) signal of the tissue during a PF time window that either temporally overlaps an excitation pulse of the pulsed optical beam or a time interval within 100 ns following the excitation pulse, and the processing circuitry is configured to generate the oxygenation state from a ratio of the DF signal and the PF signal.

(B6) In embodiments of (B5), the photodetector has a blanking interval coincident with the excitation pulse.

(B7) In any of embodiments (B4)-(B6), the processing circuitry includes control circuitry communicatively coupled to the pulsed light source and the photodetector, the control circuitry controlling sequencing of the light pulses and time-gating the photodetector.

(B8) In any of embodiments (B1)-(B7), the system further includes a radiation source configured to apply a beam of ionizing radiation to the tissue.

Changes may be made in the above methods and systems without departing from the scope of the present embodiments. It should thus be noted that the matter contained in the above description or shown in the accompanying drawings should be interpreted as illustrative and not in a limiting sense. Herein, and unless otherwise indicated the phrase “in embodiments” is equivalent to the phrase “in certain embodiments,” and does not refer to all embodiments.

As used in this specification, any appendices thereto, and the appended claims, the singular forms “a,” “an,” and “the” include plural referents unless the content clearly dictates otherwise. It should also be noted that the term “or” is generally employed in its sense including “and/or” unless the content clearly dictates otherwise. Regarding instances of the terms “and/or” and “at least one of,” for example, in the cases of “A and/or B,” “at least one of A and B,” and “at least one of A or B,” such phrasing encompasses the selection of (i) A only, or (ii) B only, or (iii) both A and B. In the cases of “A, B, and/or C,” “at least one of A, B, and C,” and “at least one of A, B, or C,” such phrasing encompasses the selection of (i) A only, or (ii) B only, or (iii) C only, or (iv) A and B only, or (v) A and C only, or (vi) B and C only, or (vii) each of A and B and C. This may be extended for as many items as are listed.

The following claims are intended to cover all generic and specific features described herein, as well as all statements of the scope of the present method and system, which, as a matter of language, might be said to fall therebetween.

What is claimed is:

1. A tissue-oxygenation monitoring method, comprising:
illuminating tissue that contains protoporphyrin IX (PPIX) with an illumination having spectral components within a fluorescence excitation band of the PPIX, thereby generating a delayed fluorescence (DF) signal propagating from the tissue;
detecting the DF signal; and
determining a tissue oxygenation state at least in part from the detected DF signal.

2. The method of claim 1, said determining comprising determining, from an intensity of the DF signal, the tissue oxygenation state as either hypoxic, not hypoxic, a degree of hypoxia, or a probability of being hypoxic.

3. The method of claim 1, said determining comprising comparing an intensity of the DF signal to a plurality of reference DF signal intensities, each mapped to a tissue oxygenation level.

4. The method of claim 1,
said illuminating further generating a prompt fluorescence (PF) signal; and
said determining comprising determining the tissue oxygenation state from a ratio of the DF signal and the PF signal.

5. The method of claim 1, the tissue being of a mammal, the method further comprising:
inducing production of PPIX in the tissue by administering aminolevulinic acid (ALA) to the mammal.

6. The method of claim 1, the illumination being a pulsed optical beam, said illuminating comprising illuminating the tissue during each of a plurality of temporal illumination intervals.

7. The method of claim 6, each pair of temporal illumination intervals being temporally separated by a respective non-illumination interval, the method further comprising:
detecting the DF signal during a DF time window that is entirely within a non-illumination interval.

8. The method of claim 1,
said detecting comprising: capturing a DF image of the tissue by detecting the DF signal with a camera; and
said determining comprising: determining the tissue oxygenation state at least in part from the DF image.

9. The method of claim 8, said illuminating further generating a prompt fluorescence (PF) signal:
said detecting comprising: capturing a PF image of the tissue by detecting the PF signal with the camera; and
said determining comprising: determining the tissue oxygenation from a ratio of the DF image and the PF image.

10. The method of claim 9,
capturing the DF image comprising capturing the PF image during even frames of the camera; and
capturing the DF image comprising capturing the DF image during odd frames of the camera.

11. The method of claim 1, further comprising applying ionizing radiation to the tissue.

12. The method of claim 11:
said illuminating comprising generating, prior to applying the ionizing radiation to the tissue, a pre-treatment DF signal; and
said determining comprising determining the tissue oxygenation state at least in part by subtracting the pre-treatment DF signal from the DF signal.

13. A tissue-oxygenation monitoring system, comprising:
a light source configured to illuminate tissue with illumination having spectral components within a fluorescence excitation band of protoporphyrin IX (PPIX), thereby generating a delayed fluorescence (DF) signal propagating from the tissue;
a photodetector configured to detect the DF signal; and
processing circuitry communicatively coupled to the photodetector and configured to determine, at least in part from the detected DF signal, an oxygenation state indicative of oxygenation of the tissue.

14. The system of claim 13,
the photodetector being a camera that detects the DF signal as a DF image; and
the processing circuitry comprising an image processor;
the processing circuitry and the image processor being configured to generate the oxygenation state at least in part from the DF image.

15. The system of claim 13, the light source being a pulsed light source and the illumination being a pulsed optical beam.

16. The system of claim 15, the photodetector being gated and synchronized to the pulsed light source, and thereby being configured to detect the DF signal during a DF time window that does not overlap with any of light pulses of the pulsed optical beam.

17. The system of claim 16,
the photodetector being further configured to detect a prompt fluorescence (PF) signal of the tissue during a PF time window that either temporally overlaps an excitation pulse of the pulsed optical beam and/or a time interval within 100 ns following the excitation pulse; and
the processing circuitry being configured to generate the oxygenation state from a ratio of the DF signal and the PF signal.

18. The system of claim 17, the photodetector having a blanking interval coincident with the excitation pulse.

19. The system of claim 16, the processing circuitry comprising:
control circuitry, communicatively coupled to the pulsed light source and the photodetector, that controls sequencing of the light pulses and time-gates the photodetector.

20. The system of claim 13, further comprising a radiation source configured to apply a beam of ionizing radiation to the tissue.
